(19) United States
(12) Patent Application Publication
(10) Pub. No.: US 2020/0306761 A1
(43) Pub. Date: Oct. 1, 2020

(54) THERMAL CYCLING SYSTEM

(71) Applicant: Delta Electronics Int'l (Singapore) Pte Ltd, Singapore (SG)

(72) Inventors: Bo Ma, Singapore (SG); Jei-Yin Yiu, Singapore (SG); Mark David Verdeflor Panoncillo, Singapore (SG)

(21) Appl. No.: 16/538,967

(22) Filed: Aug. 13, 2019

(30) Foreign Application Priority Data
Apr. 1, 2019 (SG) 10201902905U

(51) Int. Cl. B01L 7/00; C12Q 1/686

(52) U.S. Cl. CPC B01L 7/5255; C12Q 1/686; B01L 2300/1805; B01L 2300/0654; B01L 2300/1894; B01L 2300/1872

(57) ABSTRACT

A thermal cycling system for processing a bio sample includes a chamber, a photonic system and a cooling device. The photonic system includes a light-emitting unit. The light-emitting unit is configured to irradiate the bio sample for heating the bio sample rapidly. The cooling device is attached outside the chamber for cooling the bio sample inside the chamber. The bio sample is continuously cooled by the cooling device, and the light-emitting unit is selectively enabled or disabled according to a thermal cycling profile. Therefore, an ultrafast thermal cycling and a precise control of temperature are implemented.

### CROSS-REFERENCE TO RELATED APPLICATION

This application claims priority from Singapore Patent Application No. 10201902905U, filed on Apr. 1, 2019, the entire contents of which are incorporated herein by reference for all purposes.

### FIELD OF THE DISCLOSURE

The present disclosure relates to a thermal cycling system, and more particularly to a thermal cycling system applied to a polymerase chain reaction.

### BACKGROUND OF THE DISCLOSURE

The demand of fast turnaround time in quantitative polymerase chain reaction (qPCR) market becomes urgent recently. End users of qPCR systems from different fields, such as hospitals, research institutions or clinics in rural areas all eager to get the test result as soon as possible. Doctors need to get the report to identify infectious diseases that the patients might have, so the prompt treatment is necessary to save their life.

In rural areas or developing countries, ultrafast qPCR will be a solution to provide in-vitro diagnostic report in few minutes, instead of several days. However, one of the bottlenecks of ultrafast qPCR system is the speed of thermal cycling for PCR amplification. The thermal cycling technique for PCR amplification not only needs to meet the requirement of fast heating and cooling rate, but also needs to provide the precise and stable working temperature at different temperature stages during PCR amplification.

The thermal cycling technologies for the applications mentioned above could not meet the requirement of PCR amplification. A variety of thermal cycling technologies have been developed and applied for qPCR amplification in recent years, but those technologies have different drawbacks such as slow thermal cycling speed, bulky size with heavy system, and imprecise control of temperature.

Therefore, how to develop a thermal cycling system that can solve the drawbacks in prior arts, have advantages fitting the applications, is substantially the urgent problem that must be solved right now.

### SUMMARY OF THE DISCLOSURE

It is an object of the present disclosure to provide a thermal cycling system in order to overcome the above-mentioned drawbacks encountered by the prior arts.

The present disclosure provides a thermal cycling system. Since the bio sample is continuously cooled by the cooling device, and the light-emitting unit is selectively enabled or disabled according to the thermal cycling profile for heating the bio sample, an ultrafast thermal cycling is implemented. In addition, the thermal cycling system is small and light, and a precise control of temperature is achieved.

The present disclosure also provides a thermal cycling system. By utilizing the optical guiding unit with an output end matched with the chamber, a uniform heating is implemented.

In accordance with an aspect of the present disclosure, there is provided a thermal cycling system for processing a bio sample so as to perform a detection. The thermal cycling system includes a chamber, a photonic system and a cooling device. The photonic system includes a light-emitting unit. The light-emitting unit is configured to irradiate the bio sample for heating the bio sample rapidly. The cooling device is attached outside the chamber for cooling the bio sample inside the chamber. The bio sample is continuously cooled by the cooling device, and the light-emitting unit is selectively enabled or disabled according to a thermal cycling profile.

The above contents of the present disclosure will become more readily apparent to those ordinarily skilled in the art after reviewing the following detailed description and accompanying drawings, in which:

### BRIEF DESCRIPTION OF THE DRAWINGS

FIG. 1 schematically illustrates the structure of a photonic system of a thermal cycling system according to an embodiment of the present disclosure;

FIG. 2A schematically illustrates the exploded view of a chamber according to an embodiment of the present disclosure;

FIG. 2B schematically illustrates the assembled view of the chamber shown in FIG. 2A;

FIG. 3 schematically illustrates a temperature-time diagram of a test result recording thermal cycles implemented by an embodiment of a thermal cycling system of the present disclosure;

FIG. 4 schematically illustrates another temperature-time diagram of another test result recording thermal cycles implemented by another embodiment of a thermal cycling system of the present disclosure; and

FIG. 5 schematically illustrates the structure of a thermal cycling system according to an embodiment of the present disclosure.

### DETAILED DESCRIPTION OF THE PREFERRED EMBODIMENT

The present disclosure will now be described more specifically with reference to the following embodiments. It is to be noted that the following descriptions of preferred embodiments of this disclosure are presented herein for purpose of illustration and description only. It is not intended to be exhaustive or to be limited to the precise form disclosed.

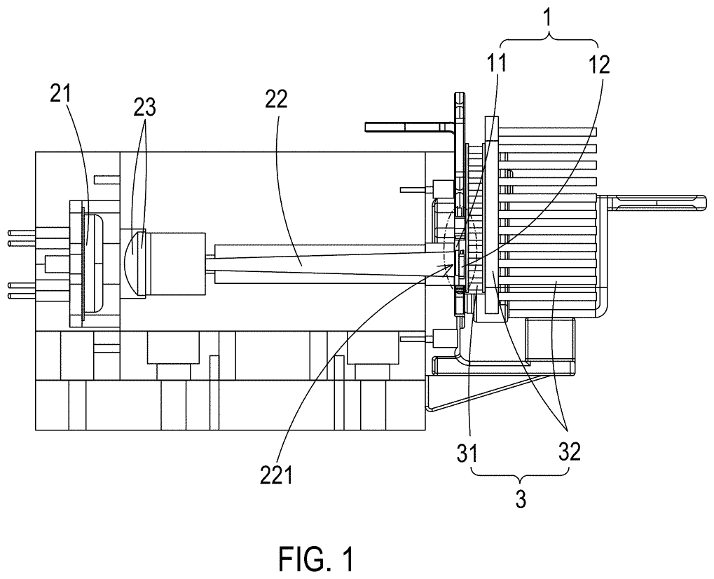

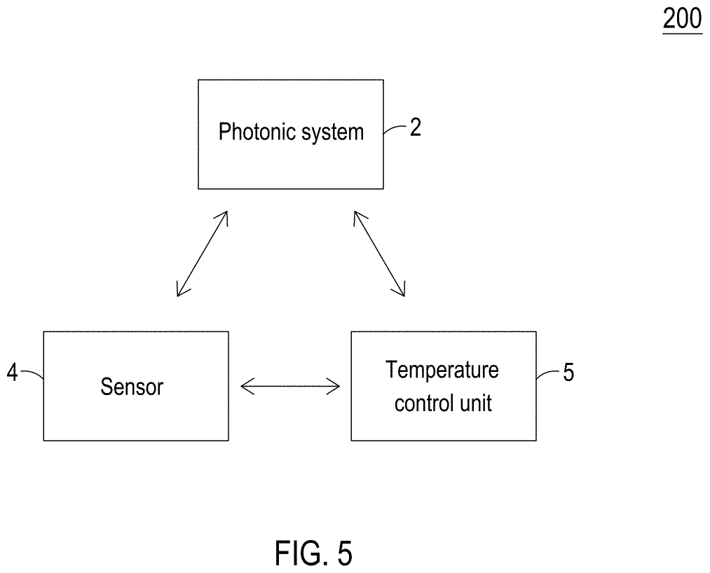

Please refer to FIG. 1 and FIG. 5. FIG. 1 schematically illustrates the structure of a photonic system of a thermal cycling system according to an embodiment of the present disclosure. FIG. 5 schematically illustrates the structure of a thermal cycling system according to an embodiment of the present disclosure. As shown in FIG. 1 and FIG. 5, a thermal cycling system of the present disclosure is used for processing a bio sample so as to perform a detection, and the thermal cycling system is preferably utilized to perform a biological detection using a quantitative polymerase chain reaction (qPCR), but not limited thereto. The thermal cycling system includes a chamber 1, a photonic system 2 and a cooling device 3. The photonic system 2 includes a light-emitting unit 21. The light-emitting unit 21 is configured to irradiate the bio sample for heating the bio sample received by the chamber 1 rapidly. The cooling device 3 is attached outside the chamber 1 for cooling the bio sample inside the chamber 1. The bio sample is continuously cooled by the cooling device 3, and the light-emitting unit 21 is selectively enabled or disabled according to a thermal cycling profile. Under the structure of the photonic system 2 of the thermal cycling system, 40 cycles of thermal cycling of the bio sample from 60 to 95 degrees Celsius are implemented within 2 minutes. While in prior art, 40 cycles of thermal cycling of a bio sample take 30-50 minutes. That is to say, an ultrafast thermal cycling is implemented by the present disclosure. In addition, the thermal cycling system is small and light due to its simple structure.

In some embodiments, according to the thermal cycling profile, the light-emitting unit 21 is selectively enabled to heat the bio sample to a first preset temperature (e.g. 95 degrees Celsius for denaturation), and when the light-emitting unit is selectively disabled, the bio sample is cooled to a second preset temperature by the cooling device 3 (e.g. 60 or 65 degrees Celsius for annealing).

Specifically, the light-emitting unit 21 is not limited to an infrared laser unit, and a wavelength of the light emitted by the light-emitting unit is in a range of 700 to 900 nanometers, preferably 808 plus or minus 3 nanometers, and a maximum power of the light-emitting unit is 30 Watts. Alternatively, the light-emitting unit 21 may be a laser LED, a tungsten lamp or a halogen lamp, but not limited thereto.

In some embodiments, light is emitted from the light-emitting unit 21 to an optical path. The photonic system 2 further includes an optical guiding unit 22 disposed on the optical path. The optical guiding unit 22 has an output end 221. The chamber 1 is disposed behind the optical guiding unit 22 along the optical path for receiving the bio sample. The light emitted by the light-emitting unit 21 is guided to the chamber 1 through the output end 221 of the optical guiding unit 22, and the chamber 1 is matched with the output end 221. The photonic system 2 includes a condensing optics 23, and the condensing optics 23 is disposed between the light-emitting unit 21 and the optical guiding unit 22 along the optical path for converging the light and enhancing the optical characteristics. Moreover, the condensing optics 23 is a condenser, a filter or a focusing lens, but not limited thereto.

In some embodiments, the optical guiding unit 22 is a homogenizer. The homogenizer is preferred to be wedge-shaped, and the light emitted by the light-emitting unit 21 is magnified and homogenized from a light beam with area equal to 2.5 mm×2.5 mm (i.e. 6.25 mm²) to a square beam with area equal to 5 mm×5 mm (i.e. 25 mm²). Certainly, other kinds of near IR transparent window materials such as quartz, MgF₂, and LiF are feasible to be used as the homogenizer. In particular, the optical guiding unit 22 is a wedge-shaped homogenizer with area increasing from one side near the light-emitting unit 21 to the other side near the chamber 1. Area of the output end 221 is larger than or equal to the area of the square beam, and a size and a shape of the chamber 1 is matched with the output end 221, such that a uniform heating is implemented.

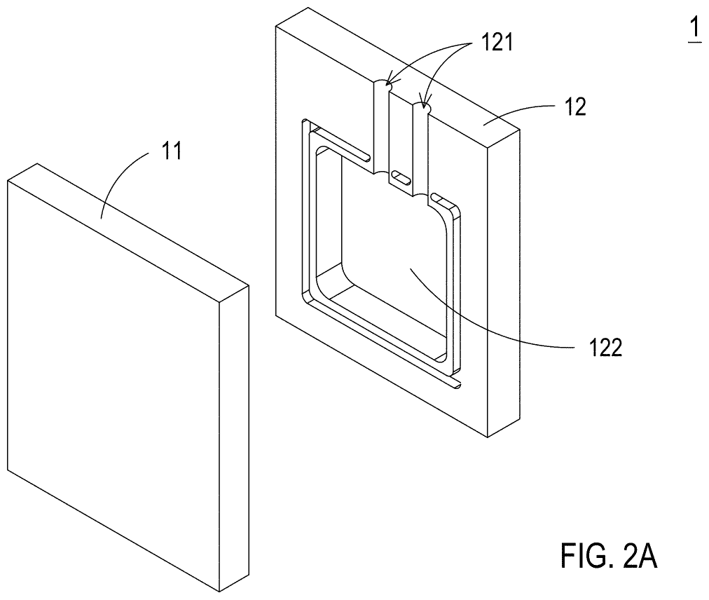

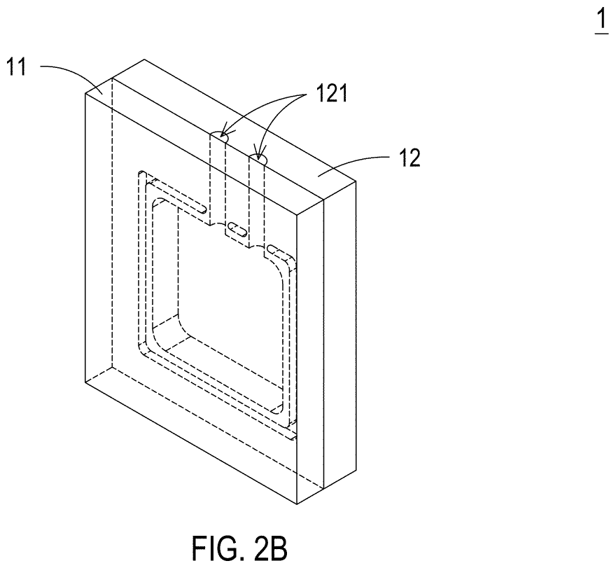

Please refer to FIG. 1, FIG. 2A, and FIG. 2B. FIG. 2A schematically illustrates the exploded view of a chamber according to an embodiment of the present disclosure. FIG. 2B schematically illustrates the assembled view of the chamber shown in FIG. 2A. As shown in FIGS. 1, 2A, and 2B, the chamber 1 of the photonic system 2 of the thermal cycling system includes a protection plate 11 and a main body 12. The protection plate 11 is disposed between the output end 221 and the main body 12. The protection plate 11 is not limited to a glass plate. The thickness of the protection plate 11 can be in a range from 0.5 to 1 mm, but not limited thereto. The material of the protection plate 11 can be a near IR transparent material such as quartz, MgF₂, or LiF. The main body 12 is not limited to a thermal conductive polymer, which can be a CNC machined or injection-molded.

In an embodiment, an in-plane thermal conductivity of the main body 12 is at least 24 W/mK, and a through-plane thermal conductivity of the main body 12 is at least 4.5 W/mK, in which W stands for Watts, m stands for mass, and K stands for absolute temperature. On the other hand, the color of the main body 12 must be black for infrared absorption.

In some embodiments, the main body 12 has a plurality of communication channels 121 and a recess 122. The plurality of communication channels 121 are in fluid communication with the recess 122, and the recess 122 and the plurality of communication channels 121 are covered by the protection plate 11. Preferably, the protection plate 11 and the main body 12 are compatible with the bio sample to avoid of destroying the bio sample during heating and cooling. In some embodiments, the protection plate 11 is preferred to be optically transparent, such that IR light can pass through without energy loss.

Please refer to FIG. 1 again. As shown in FIG. 1, the cooling device 3 of the thermal cycling system includes at least one active cooler 31 and at least one passive cooler 32, and the active cooler 31 and the passive cooler 32 are connected with each other.

In an embodiment, the active cooler 31 is a thermoelectric cooler, a cooling fan, a blower or forced liquid coolant, but not limited thereto. In an embodiment, the passive cooler 32 is a heatsink, a heat spreader, a heat pipe or a thermal interface material, but not limited thereto.

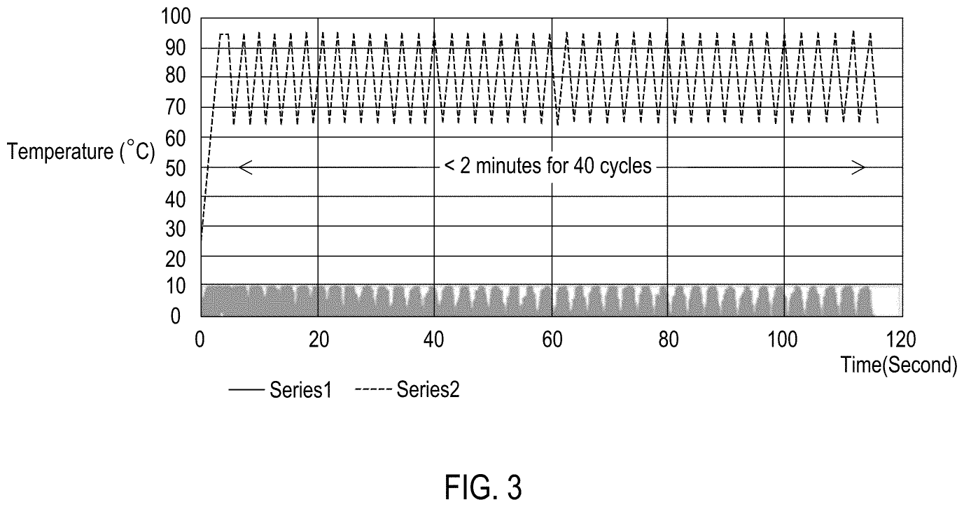

Please refer to FIG. 3. FIG. 3 schematically illustrates a temperature-time diagram of a test result recording thermal cycles implemented by an embodiment of a thermal cycling system of the present disclosure. As shown in FIG. 3, 40 cycles of thermal cycling of the bio sample from 65 to 95 degrees Celsius are implemented within 2 minutes (i.e. 120 seconds). In this test, the volume of the bio sample is 25 to 37.8 microliters. The heating rate is about 20 degrees Celsius per second, and the cooling rate is about 30 degrees Celsius per second. Series 1 indicates an IR laser chosen as the light-emitting unit. Series 2 indicates the bio sample.

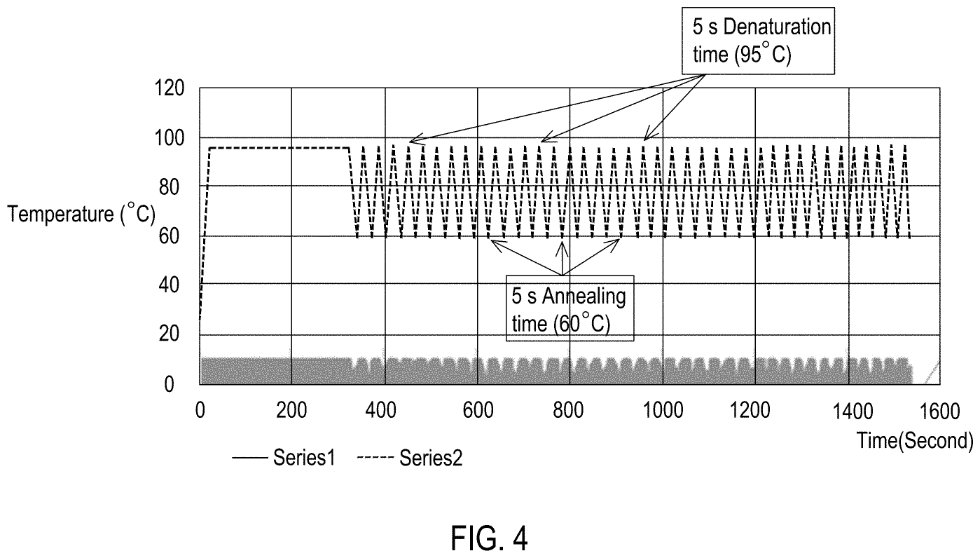

Please refer to FIG. 4. FIG. 4 schematically illustrates another temperature-time diagram of another test result recording thermal cycles implemented by another embodiment of a thermal cycling system of the present disclosure. As shown in FIG. 4, to perform a precise control of temperature, the thermal cycling system of the present disclosure accurately controls a holding temperature within 1 degree Celsius at the denaturation and annealing stage in thermal cycling process. It avoids the fluctuation of temperature in ambient environment and stabilizes the polymerase chain reaction (PCR) amplification process of the bio sample. In this test, the holding time is 300 seconds, and the holding temperature is 95 degrees Celsius. The holding temperature is only fluctuated within 95 plus or minus 1 degree in 120 seconds. To make the thermal cycling stable, the denaturation time and the annealing time are each set to be 5 seconds. The total time for 40 cycles is approximately 25.67 minutes. In this test, a precise control of temperature is achieved.

In some embodiments, the details of the thermal cycling profile include the steps of (a) heating the bio sample to a first preset temperature at a heating rate, (b) maintaining the first preset temperature for an initial holding time, (c) cooling the bio sample to a second preset temperature at a cooling rate, (d) maintaining the second preset temperature for a second holding time, (e) heating the bio sample to the first preset temperature at the heating rate, (f) maintaining the first preset temperature for a first holding time, and (g) repeating the steps (c) to (f) for a predetermined number of cycles.

In some embodiments, the thermal cycling profile can be configured with P, I, D control based on requirements. In other words, parameters such as PID control, holding time, laser output power, sampling time, holding temperature, ramping rate, variation of temperature, number of cycles, input power of cooling unit, . . . , etc. are configurable.

Please refer to FIG. 1 and FIG. 5 again. As shown in FIG. 1 and FIG. 5, a thermal cycling system 200 according to an embodiment of the present disclosure further includes a sensor 4 and a temperature control unit 5. The sensor 4 is connected with a computer for monitoring a real-time temperature of the bio sample and an output power of the light-emitting unit 21. The temperature control unit 5 is connected with the light-emitting unit 21 and the cooling device 3, and the light-emitting unit 21 and the cooling device 3 are controlled by the temperature control unit 5 according to the real-time temperature and the output power sensed by the sensor 4, and the thermal cycling profile.

In an embodiment, the temperature control unit 5 includes at least one thermocouple, infrared sensor or camera.

From the above discussion, the present disclosure provides a thermal cycling system. Since the bio sample is continuously cooled by the cooling device, and the light-emitting unit is selectively enabled or disabled according to the thermal cycling profile for heating the bio sample, an ultrafast thermal cycling is implemented. In addition, the thermal cycling system is small and light, and a precise control of temperature is achieved. Meanwhile, by utilizing the optical guiding unit with an output end matched with the chamber, a uniform heating is implemented.

The present disclosure can be modified by one skilled in the art as various modifications, but none of the modifications is not included within the scope of the claims.

What is claimed is:

1. A thermal cycling system for processing a bio sample so as to perform a detection, comprising:
a chamber for accommodating the bio sample;
a photonic system, comprising:
a light-emitting unit which is configured to irradiate the bio sample for heating the bio sample rapidly; and
a cooling device attached outside the chamber for cooling the bio sample inside the chamber,
wherein the bio sample is continuously cooled by the cooling device, and the light-emitting unit is selectively enabled or disabled according to a thermal cycling profile.

2. The thermal cycling system according to claim 1, wherein according to the thermal cycling profile, the light-emitting unit is selectively enabled to heat the bio sample to a first preset temperature, and when the light-emitting unit is selectively disabled, the bio sample is cooled to a second preset temperature by the cooling device.

3. The thermal cycling system according to claim 1 further comprising a sensor and a temperature control unit, wherein the sensor is connected with a computer for monitoring a real-time temperature of the bio sample and an output power of the light-emitting unit, the temperature control unit is connected with the light-emitting unit and the cooling device, and the light-emitting unit and the cooling device are controlled by the temperature control unit according to the real-time temperature and the output power sensed by the sensor, and the thermal cycling profile.

4. The thermal cycling system according to claim 1, wherein the light-emitting unit is an infrared laser unit, and a wavelength of the light emitted by the light-emitting unit is in a range of 700 to 900 nanometers.

5. The thermal cycling system according to claim 1, wherein the light-emitting unit is a laser LED, a tungsten lamp or a halogen lamp.

6. The thermal cycling system according to claim 1, wherein light is emitted from the light-emitting unit to an optical path, the photonic system further comprises an optical guiding unit disposed on the optical path, the optical guiding unit has an output end, the chamber is disposed behind the optical guiding unit along the optical path, the light is guided to the chamber through the output end, and the chamber is matched with the output end.

7. The thermal cycling system according to claim 6, wherein the photonic system further comprises a condensing optics, and the condensing optics is disposed between the light-emitting unit and the optical guiding unit along the optical path.

8. The thermal cycling system according to claim 7, wherein the condensing optics is a condenser or a focusing lens.

9. The thermal cycling system according to claim 6, wherein the optical guiding unit is a homogenizer.

10. The thermal cycling system according to claim 9, wherein the homogenizer is wedge-shaped, and the light emitted by the light-emitting unit is magnified and homogenized from a light beam with area equal to 2.5 mm×2.5 mm to a square beam with area equal to 5 mm×5 mm.

11. The thermal cycling system according to claim 10, wherein area of the output end is larger than or equal to the area of the square beam, and a size and a shape of the chamber is matched with the output end.

12. The thermal cycling system according to claim 6, wherein the chamber comprises a protection plate and a main body, and the protection plate is disposed between the output end and the main body.

13. The thermal cycling system according to claim 12, wherein the protection plate is a glass plate with thickness in a range from 0.5 to 1 mm, and the main body is a thermal conductive polymer.

14. The thermal cycling system according to claim 12, wherein an in-plane thermal conductivity of the main body is at least 24 W/mK, and a through-plane thermal conductivity of the main body is at least 4.5 W/mK.

15. The thermal cycling system according to claim 12, wherein a color of the main body is black for infrared absorption.

16. The thermal cycling system according to claim 12, wherein the main body has a recess and a plurality of communication channels, the plurality of communication channels are in fluid communication with the recess, and the recess and the plurality of communication channels are covered by the protection plate.

17. The thermal cycling system according to claim 1, wherein 40 cycles of thermal cycling of the bio sample from 60 to 95 degrees Celsius are implemented within 2 minutes.

18. The thermal cycling system according to claim 1, wherein the cooling device comprises at least one active cooler and at least one passive cooler.

19. The thermal cycling system according to claim 18, wherein the passive cooler is a heatsink, a heat spreader, a heat pipe or a thermal interface material.

20. The thermal cycling system according to claim 18, wherein the active cooler is a thermoelectric cooler, a cooling fan, a blower or forced liquid coolant.

21. The thermal cycling system according to claim 1, wherein the thermal cycling system is utilized to perform a biological detection using a quantitative polymerase chain reaction.
